(19) United States
(12) Patent Application Publication
(10) Pub. No.: US 2021/0022824 A1
(43) Pub. Date: Jan. 28, 2021

(54) SURGICAL SAFETY TOWEL

(71) Applicants: Christopher A. Murray, Orange, CA (US); Jeffrey E. Bowles, Redondo Beach, CA (US)

(72) Inventors: Christopher A. Murray, Orange, CA (US); Jeffrey E. Bowles, Redondo Beach, CA (US)

(21) Appl. No.: 16/983,385

(22) Filed: Aug. 3, 2020

Related U.S. Application Data
(63) Continuation-in-part of application No. 16/841,409, filed on Apr. 6, 2020.
(60) Provisional application No. 62/829,760, filed on Apr. 5, 2019.

(51) Int. Cl. A61B 50/37; A61B 90/00; A61B 90/90; A61B 50/20

(52) U.S. Cl. CPC A61B 50/37; A61B 90/08; A61B 50/20; A61B 90/90; A61B 90/39

(57) ABSTRACT

A surgical towel which can be used in any surgical procedure to deposit samples from procedures such as hysterectomies, orthopedic and breast biopsies. The surgical safety towels are sterile and have markings thereon which facilitate identification and categorization of the collected tissue sample. The surgical safety towel preferably includes discrete areas for placing tissue samples from different parts of the body. In one embodiment, the surgical safety towel has an area marked left and an area marked right which may be used to segregate tissue samples taken from the left or right side of the body. Additionally, the surgical safety towels desirably have length markings on at least one side which provide reference indicators against which the tissue samples may be measured and photographed.

### FIELD OF THE INVENTION

The present application is a continuation-in-part of U.S. application Ser. No. 16/841,409, filed Apr. 6, 2020, which claims priority under 35 U.S.C. § 119 to U.S. Provisional Application Ser. No. 62/829,760, filed Apr. 5, 2019.

### FIELD OF THE INVENTION

The present invention is directed to a surgical towel used to deposit samples from procedures such as; but not limited to hysterectomies, orthopedics, lung resection, mastectomies and breast biopsies.

### BACKGROUND OF THE INVENTION

In medicine, sampling, such as biopsy, is gathering of matter from the body to aid in the process of a medical diagnosis and/or evaluation of an indication for treatment, further medical tests or other procedures. Tissue samples are often taken from the breast, ovaries and other samples, during mastectomies, hysterectomies, orthopedics, lung resection and in bariatric procedures, to name just a few. Examples of biopsy procedures are for sampling bone marrow, brain tissue, breast tissue, skin and liver tissues. Since fluid content in samples could influence results, such as weight, they may be placed on an absorbent paper towel so that any excess fluid is absorbed. Fresh tissue may be placed on a sterile towel, in a Petri dish, or in a sterile jar. Sometimes the tissue sample is placed on a moist towel so that the tissue does not dry out.

Although tissue sampling is not a new science, there remains a need for a more efficient way for collecting and categorizing tissue samples.

### SUMMARY OF THE INVENTION

The present application provides a number of surgical safety towels on which tissue samples may be placed immediately after extraction. The surgical safety towels are sterile and have markings thereon which facilitate identification and categorization of the collected tissue sample. More specifically, the fabric is gas sterilized such as with ethylene oxide (ETO) after having the markings printed thereon such that the markings are sterile, and then packaged in a manner to maintain sterility. The surgical safety towel preferably includes discrete areas for placing tissue samples from different parts of the body. In one embodiment, the surgical safety towel has an area marked left and an area marked right which may be used to segregate tissue samples taken from the left or right side of the body. Additionally, the surgical safety towels desirably have length markings on at least one side which provide reference indicators against which the tissue samples may be measured and photographed.

The surgical safety towel will benefit the surgical team by drastically reducing the mistakes that can occur during procedures. The dual-purpose measurement and L/R demarcation will assist not only the surgeon but the technician in unnecessary mistakes. Often, surgeons request a specific measurement for a suture length. The optimal surgical safety towel addresses this demand with the handy length measurements and the capacity for measuring both inches and centimeters. The towel will assist the technician and nurses when noting which part of the body the specimen has been removed and the specimen can be photographed showing the size with measurements feature.

Features of the surgical towel include:

1) indexed length markings, preferably along both longitudinal edges of the rectangular towel, and preferably in both English and metric units.

2) two relatively large demarked areas with left (L) and right (R) indicators, optional circular area, to provide places to put down specimens taken from the body such as from the breast, ovaries, lungs, and in bariatric procedures.

3) the length markings and specimen areas are first printed on the towels which are then gas sterilized.

A further understanding of the nature and advantages of the invention will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 is a first embodiment of a surgical safety towel having L/R indicators within large circles and length measurements along two opposite sides;

FIG. 2 is a second embodiment of a surgical safety towel having L/R indicators within large circles and length measurements along two opposite sides;

FIG. 3A is a third embodiment of a surgical safety towel having L/R indicators within large circles and length measurements along two opposite sides;

FIG. 3B is a modification of the third embodiment of a surgical safety towel having L/R indicators and length measurements along two opposite sides;

FIG. 4 is a fourth embodiment of a surgical safety towel having L/R indicators within smaller circles and length measurements along two adjacent sides;

FIG. 5 is a fifth embodiment of a surgical safety towel having L/R indicators within smaller circles and length measurements along two adjacent sides;

FIG. 6 is a sixth embodiment of a surgical safety towel having L/R indicators within smaller circles and length measurements along two opposite sides;

FIG. 7 is a seventh embodiment of a surgical safety towel having L/R indicators within small circles and length measurements along one side; and

FIGS. 8A and 8B are complementary versions of an eighth embodiment of a surgical safety towel having either a large left (L) or right (R) indicator in a central position and length measurements along two opposite sides.

### DETAILED DESCRIPTION OF THE PREFERRED EMBODIMENTS

The present application is directed to a surgical safety towel having discrete areas for placing tissue samples from different parts of the body. The towel is made of a sterile fabric that is gas sterilized after having markings printed thereon. The towels illustrated herein have a rectangular periphery with two parallel long edges and two parallel short edges. Different shapes of towels are contemplated, and the term peripheral edge should be understood to mean an outer edge, regardless of orientation or shape.

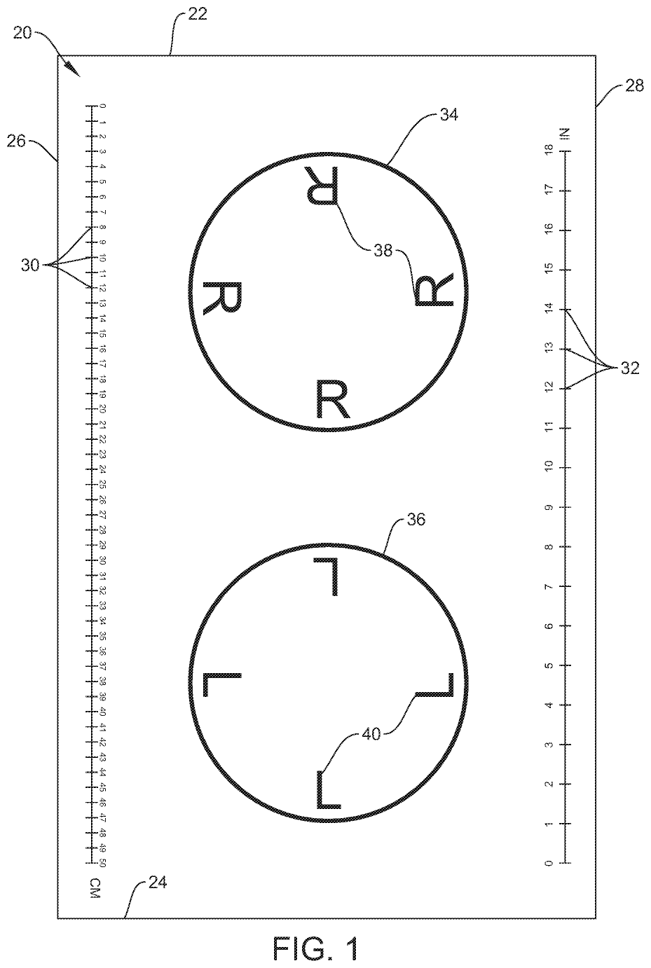

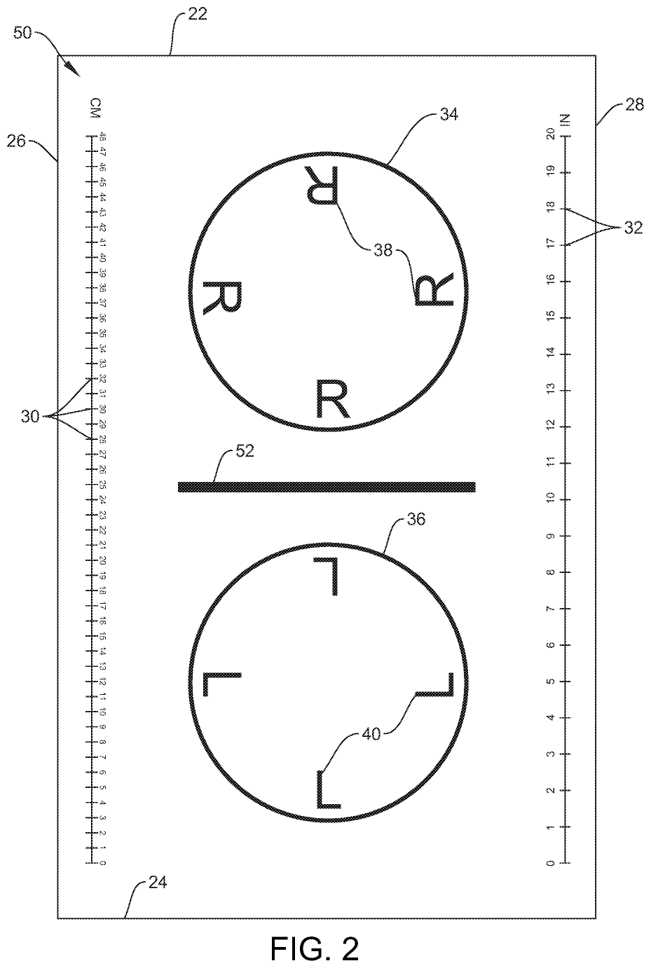

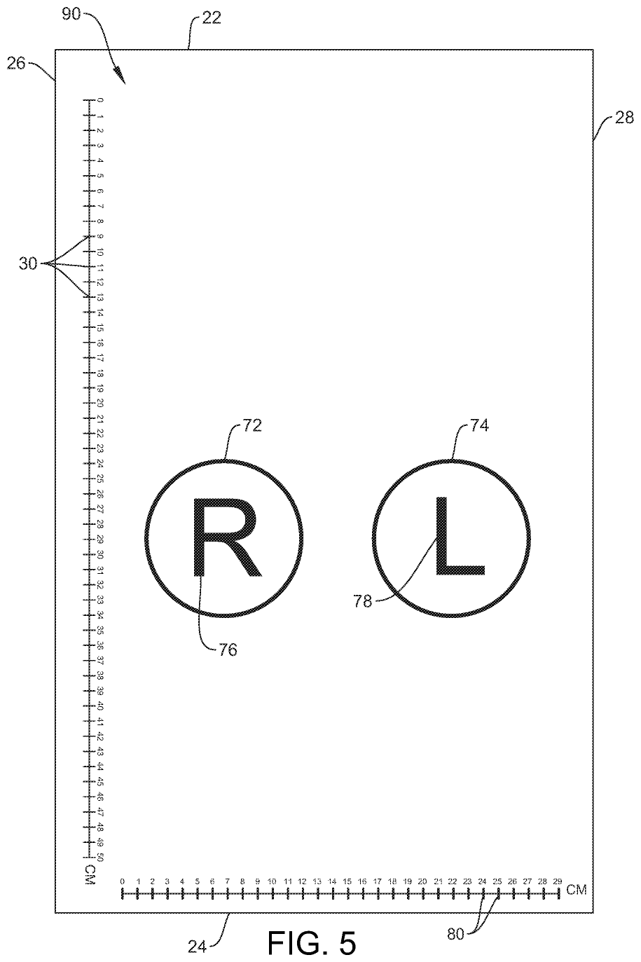

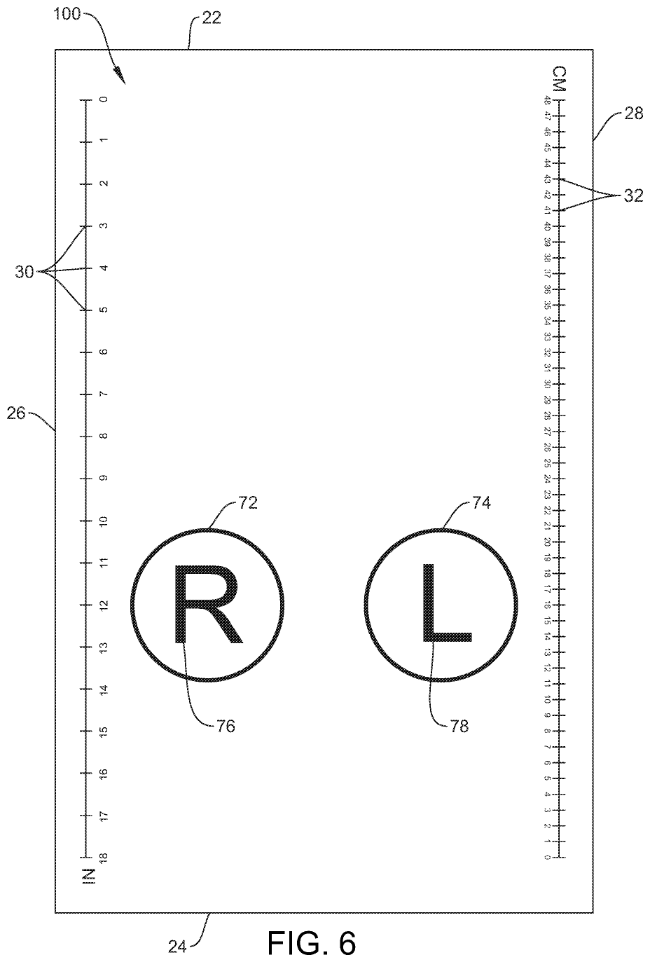

A number of primary embodiments are disclosed herein, and various differences are described, and it should be understood that that any one compatible feature in one embodiment can be substituted into the other, and vice versa. For instance, the 4 letter markings in quadrants in the version of FIGS. 1-2 may be incorporated into the version of FIGS. 5-6, among many possible permutations.

FIG. 1 is a first embodiment of a surgical safety towel 20 having L/R indicators within large circles and length measurements along two long edges. The towel 20 is preferably in the shape of a rectangle, with a short top edge 22 opposite a short bottom edge 24, and two longer parallel left and right edges 26, 28. Centimeter and inch distance markings 30 are provided adjacent and parallel to the left edge 26 and distance markings 32 are provided adjacent and parallel to the right edge 28. In the illustrated embodiment, the distance markings 30 are metric (e.g., cm) while the opposite distance markings 32 are in English units (e.g., inches).

A first large sample circle 34 is marked in a top half of the towel 20, while a second large sample circle 36 is marked in a bottom half of the towel. Right (R) and left (L) indicator markings 38, 40 are provided within the sample circles 34, 36. In the illustrated embodiment, the indicator markings 38, 40 comprise the capital letters R and L arrayed evenly around the inside of the sample circles 34, 36 in quadrants. The capital letters R and L are oriented so as to be seen right-side up when looking at each quadrant of the circles.

FIG. 2 is a second embodiment of a surgical safety towel 50 having L/R indicators within large circles and length measurements along two long edges. As with the earlier-described towel 20 (and with all of the towels described herein), the towel 50 is in the shape of a rectangle, with a short top edge 22 opposite a short bottom edge 24, and two longer parallel left and right edges 26, 28. The rectangular layout will not be further described, and it should be noted that the towel is easily folded into sections both lengthwise and laterally.

Once again, distance markings 30 are provided adjacent and parallel to the left edge 26 and distance markings 32 are provided adjacent and parallel to the right edge 28. In the illustrated embodiment, the distance markings 30 are metric (e.g., cm) while the opposite distance markings 32 are in English units (e.g., inches).

Also, as before, a first large sample circle 34 is marked in a top half of the towel 20, while a second large sample circle 36 is marked in a bottom half of the towel. Right (R) and left (L) indicator markings 38, 40 are provided within the sample circles 34, 36. In the illustrated embodiment, the indicator markings 38, 40 comprise the capital letters R and L arrayed evenly around the inside of the sample circles 34, 36 in quadrants. The capital letters R and L are oriented so as to be seen right-side up when looking at each quadrant of the circles.

The towel 50 further includes a thick dividing line marker 52 which clearly separates the sample circles 34, 36. This helps prevent any spillover from one circle to the other. The dividing line marker 52 is located at a longitudinal midpoint of the rectangular surgical safety towel 50 parallel to and between the shorter top and bottom edges 22, 24.

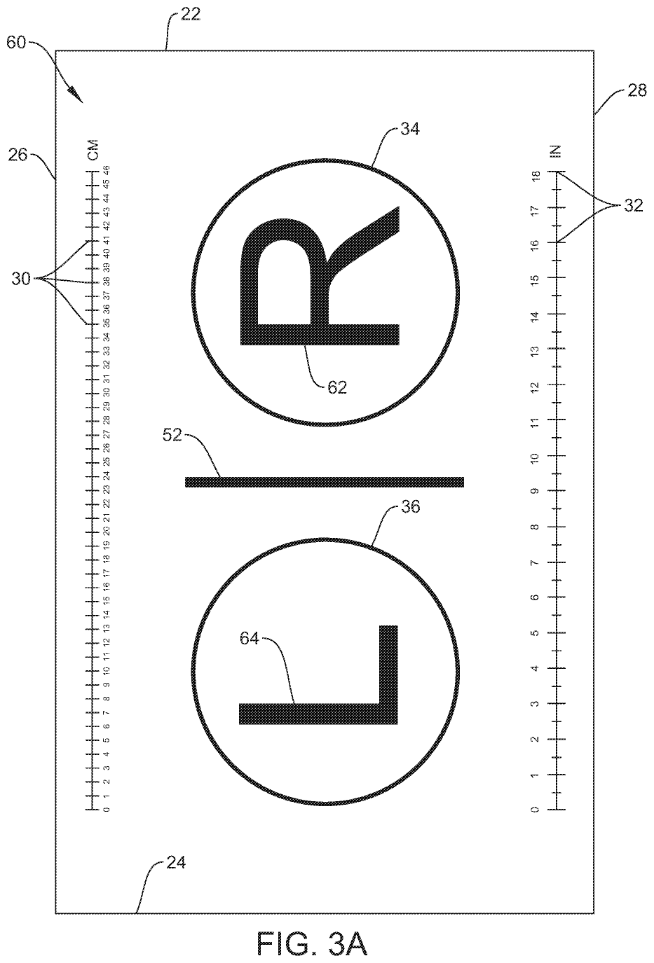

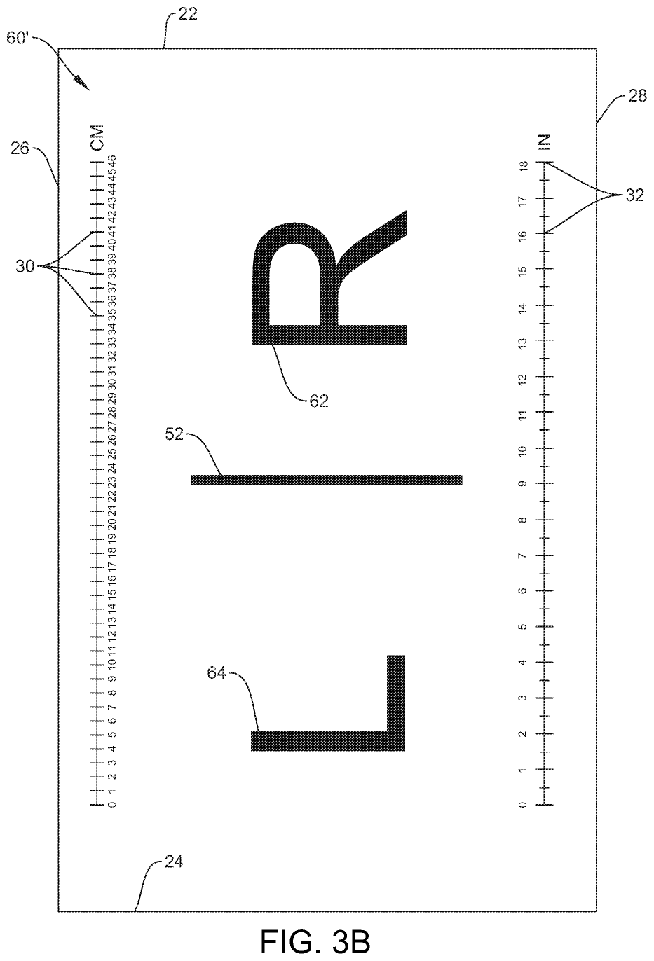

FIG. 3A is a third embodiment of a surgical safety towel 60 having L/R indicators within large circles and length measurements along two long edges. The towel 60 again has left and right distance markings 30, 32, and large sample circles 34, 36 in the top and bottom halves. Right (R) and left (L) indicator markings 62, 64 are provided within the sample circles 34, 36, but in this case, they are relatively large capital letters which fill the space within the circles 34, 36. The right (R) and left (L) indicator markings 62, 64 are oriented so that they may be read upright when the towel is rotated so that the right edge 28 is at the bottom. That is, the letters on the towel 60 read from left-to-right along the long dimension of the rectangle. Again, the distance markings 30 are metric (e.g., cm) while the opposite distance markings 32 are in English units (e.g., inches). The dividing line marker 52 is again located at a midpoint of the rectangular surgical safety towel 50 parallel to and between the shorter top and bottom edges 22, 24.

FIG. 3B is a modification of the third embodiment of a surgical safety towel 60′ having L/R indicators and length measurements along two long edges. The L/R indicators are displayed on the towel 60 without the large circles as in FIG. 3A. The towel 60 again has left and right distance markings 30, 32, and large sample circles 34, 36 in the top and bottom halves. Right (R) and left (L) indicator markings 62, 64 are provided within the sample circles 34, 36, but in this case, they are relatively large capital letters which fill the space within the circles 34, 36. Again, the right (R) and left (L) indicator markings 62, 64 are oriented so that they may be read upright when the towel is rotated so that the right edge 28 is at the bottom. Or in other words, the letters on the towel 60 read from left-to-right along the long dimension of the rectangle.

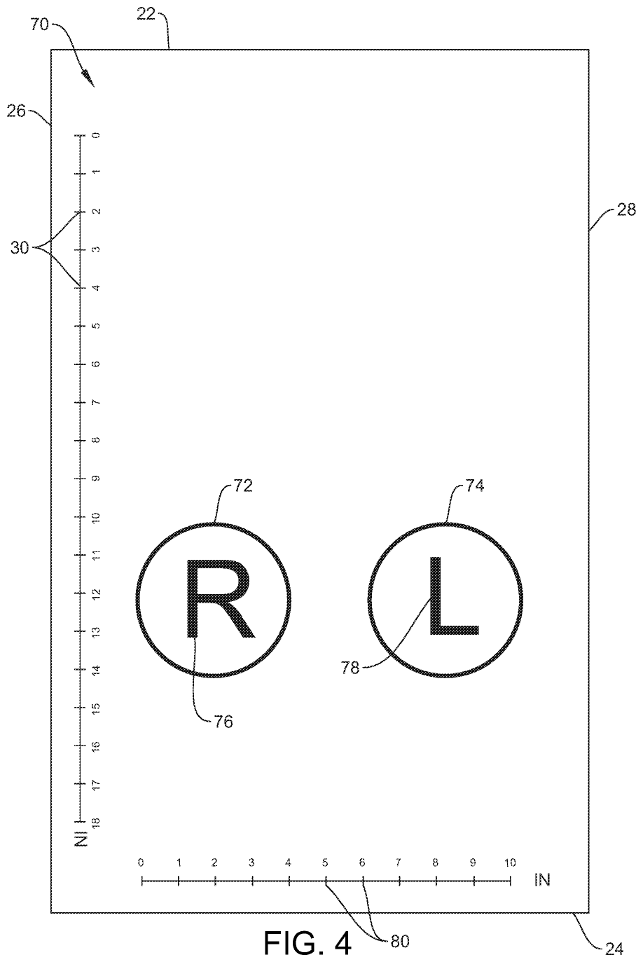

FIG. 4 is a fourth embodiment of a surgical safety towel 70 having L/R indicators within smaller circles and length measurements along two adjacent edges. The towel 70 has distance markings 30, 80 both in the same units (English) parallel to adjacent perpendicular edges 26, 24. Right (R) and left (L) indicator markings 76, 78 are provided within the smaller sample circles 72, 74, in the form of relatively large capital letters which fill the space within the circles 72, 74. The smaller circles 72, 74 are located in a lower half of the towel 70. The letter markings 76, 78 are upright when viewed from the bottom edge 24, though they may also be provided in any orientation.

FIG. 5 is a fifth embodiment of a surgical safety towel 90 having L/R indicators within smaller circles 72, 74 and length measurements 30, 80 along two adjacent perpendicular edges 24, 26. In this embodiment, the length measurements 30, 80 are both in the same units, for example metric units. The smaller circles 72, 74 are located in a lower half of the towel 90. Right (R) and left (L) indicator markings 76, 78 are provided within the respective circles 72, 74. The (R) and left (L) indicator markings may be single capital letters as shown, and may be provided in any orientation. In the example the letters may be read from the bottom edge 24.

FIG. 6 is a sixth embodiment of a surgical safety towel 100 which is essentially identical to the just-described towels 70, 90, but the length measurements 30, 32 are again adjacent and parallel to the long edges 26, 28 and in different units.

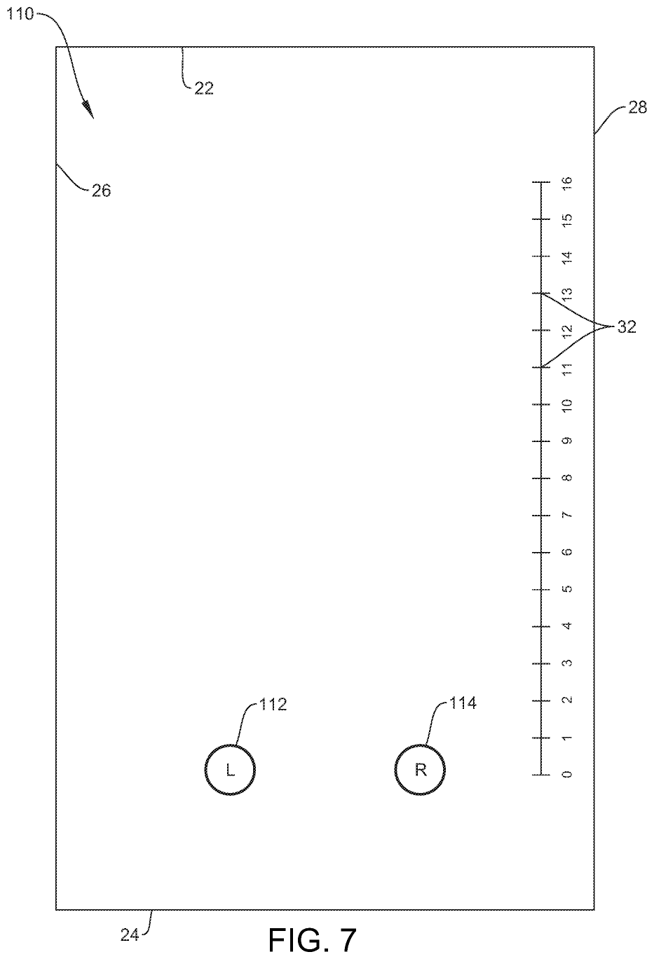

Finally, FIG. 7 is a seventh embodiment of a surgical safety towel 110 having L/R indicators within small circles 112, 114 and length measurements 32 along one only edge. The length measurements are again either in metric or English units as desired.

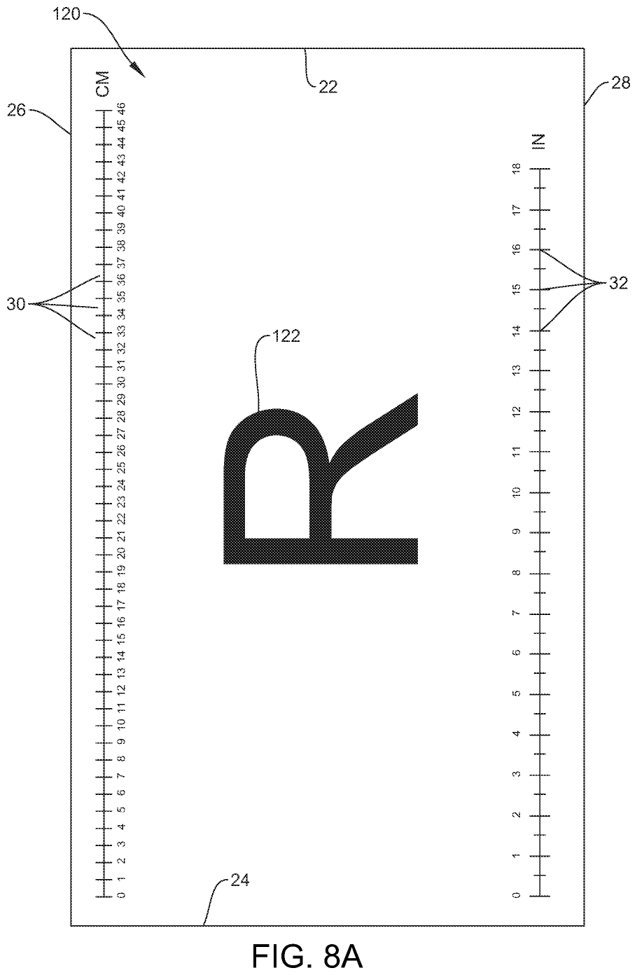

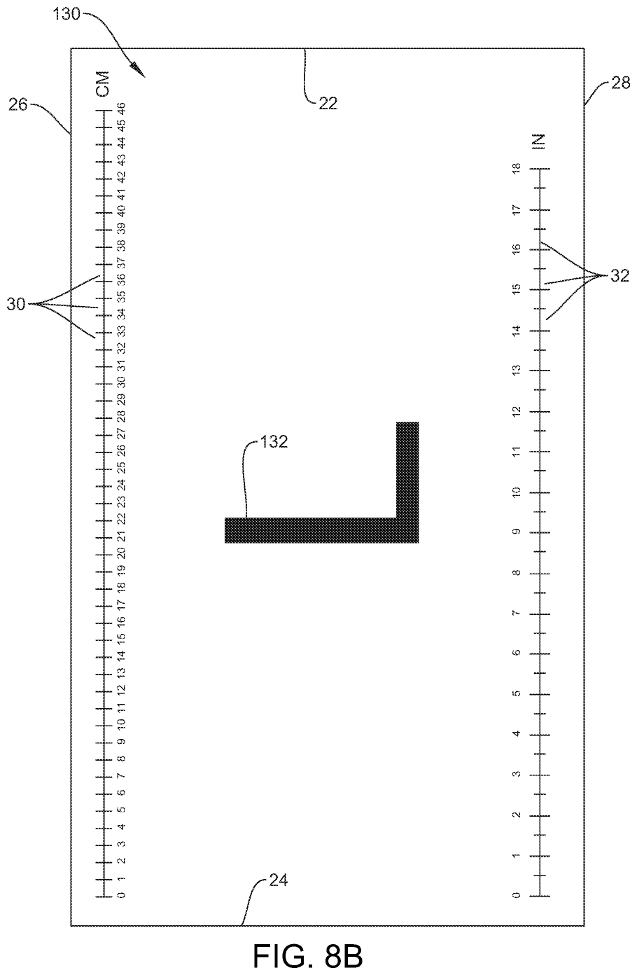

FIGS. 8A and 8B are complementary versions of an eighth embodiment of a surgical safety towel having either a large left (L) or right (R) indicator in a central position and length measurements along two opposite edges.

The towel 120 in FIG. 8A again has centimeter and inch distance markings 30, 32, and a Right (R) indicator marking 122 is provided as a relatively large capital letter generally centered on the towel 120. If the towel 120 has L×W size of 20×12 inches, the Right (R) indicator marking 122 may be 7 inches tall and 5 inches wide. Other proportional sizes are contemplated.

The towel 130 in FIG. 8B also has centimeter and inch distance markings 30, 32, and a Left (L) indicator marking 132 is provided as a relatively large capital letter generally centered on the towel 120. If the towel 120 has L×W size of 20×12 inches, the Left (L) indicator marking 132 may be 7 inches tall and 5 inches wide. Other proportional sizes are contemplated.

Importantly, the fabric of any of the towels configured as disclosed herein is gas sterilized such as with ethylene oxide (ETO) after having the markings printed thereon such that the markings are sterile, and then the towel is packaged in a manner to maintain sterility. The finished towel is sterile to the extent required by hospital procedures. This is in contrast to fabricating a sterile towel and then placing markings on it, which could negate the sterility of the towel.

Upon reading this disclosure, those skilled in the art will appreciate that various changes and modifications may be made to the preferred embodiments of the invention and that such changes and modifications may be made without departing from the spirit of the invention. Therefore, the invention in its broader aspects is not limited to the specific details, representative devices, and illustrative examples shown and described. Accordingly, departures may be made from such details without departing from the spirit or scope of the general inventive concept.

What is claimed is:

1. A surgical safety towel, comprising:
a fabric towel having peripheral edges and printed markings on one face, the markings including:
discrete areas including a first area with a letter L (left) marking and a second area with a letter R (right) marking indicating where to place and segregate tissue samples taken from a left or right side of the body, and
length markings adjacent and parallel to at least one peripheral edge which provide size reference indicators, wherein
the fabric is gas sterilized after having the markings printed thereon such that the markings are sterile.

2. The surgical safety towel of claim 1, wherein the markings further include a circle around each of the first area and second area large enough to encompass the letter L (left) marking and the letter R (right) marking, respectively.

3. The surgical safety towel of claim 1, wherein the towel has a rectangular peripheral shape with a top edge opposite a bottom edge and two parallel left and right edges both longer than the top and bottom edges, and wherein the letter L (left) marking and the letter R (right) marking are oriented to be read from left-to-right along a long dimension of the rectangular peripheral shape.

4. The surgical safety towel of claim 1, wherein the towel has a rectangular peripheral shape with a top edge opposite a bottom edge and two parallel left and right edges both longer than the top and bottom edges, and wherein the letter L (left) marking and the letter R (right) marking are oriented to be read from left-to-right along a short dimension of the rectangular peripheral shape

5. The surgical safety towel of claim 1, wherein there are two sets of length markings adjacent and parallel to two of the peripheral edges.

6. The surgical safety towel of claim 4, wherein the two sets of length markings are adjacent and parallel to opposite parallel edges.

7. The surgical safety towel of claim 4, wherein the two sets of length markings are adjacent and parallel to adjacent perpendicular edges.

8. The surgical safety towel of claim 4, wherein the two sets of length markings are in different measurement systems.

9. The surgical safety towel of claim 1, wherein the letter L (left) marking and the letter R (right) marking are each surrounded by a circle marking, and there are more than one letter L (left) marking and more than one letter R (right) marking within each circle marking.

10. The surgical safety towel of claim 9, wherein the letter L (left) markings and letter R (right) markings within each circle marking are provided in each of four quadrants within the circle marking facing outward in four different directions.

11. A surgical safety towel, comprising:
a fabric towel having peripheral edges and printed markings on one face, the markings including:
discrete areas including either a letter L (left) marking or a letter R (right) marking indicating where to place and segregate tissue samples taken from either a left or right side of the body, and
two sets of length markings adjacent and parallel to two of the peripheral edges which provide size reference indicators, wherein
the fabric is gas sterilized after having the markings printed thereon such that the markings are sterile.

12. The surgical safety towel of claim 11, wherein the markings further include a circle large enough to encompass the letter L (left) marking or the letter R (right) marking, whichever is on the towel.

13. The surgical safety towel of claim 11, wherein the towel has a rectangular peripheral shape with a top edge opposite a bottom edge and two parallel left and right edges both longer than the top and bottom edges, and wherein the letter L (left) marking or the letter R (right) marking is oriented to be read from left-to-right along a long dimension of the rectangular peripheral shape.

14. The surgical safety towel of claim 11, wherein the towel has a rectangular peripheral shape with a top edge opposite a bottom edge and two parallel left and right edges both longer than the top and bottom edges, and wherein the letter L (left) marking or the letter R (right) marking is oriented to be read from left-to-right along a short dimension of the rectangular peripheral shape

15. The surgical safety towel of claim 11, wherein the two sets of length markings are adjacent and parallel to opposite parallel edges.

16. The surgical safety towel of claim 11, wherein the two sets of length markings are adjacent and parallel to adjacent perpendicular edges.

17. The surgical safety towel of claim 11, wherein the two sets of length markings are in different measurement systems.

18. The surgical safety towel of claim 11, wherein the two sets of length markings are in the same measurement system.

19. The surgical safety towel of claim 11, wherein the letter L (left) marking or the letter R (right) marking is surrounded by a circle marking, and there are more than one letter L (left) marking or more than one letter R (right) marking within the circle marking.

20. The surgical safety towel of claim 19, wherein the letter L (left) markings or the letter R (right) markings within the circle marking are provided in each of four quadrants within the circle marking facing outward in four different directions.
